(19) United States
(12) Patent Application Publication
(10) Pub. No.: US 2019/0314436 A1
(43) Pub. Date: Oct. 17, 2019

(54) PHARMACEUTICAL COMPOSITION FOR PREVENTION OR TREATMENT OF LIVER CANCER AND HEALTH FUNCTIONAL FOOD

(71) Applicants: KOREA BIO MEDICAL SCIENCE INSTITUTE CO., LTD, Gyeongsangbuk-do (KR); THE CATHOLIC UNIVERSITY OF KOREA INDUSTRY-ACADEMIC COOPERATION FOUNDATION, Seoul (KR)

(72) Inventors: Sung-Yeoun HWANG, Incheon (KR); Seung-Kew YOON, Seoul (KR); Jung-Hee KIM, Seoul (KR)

(21) Appl. No.: 16/347,641

(22) PCT Filed: Nov. 4, 2016
(86) PCT No.: PCT/KR2016/012643

§ 371 Date: May 6, 2019

(30) Foreign Application Priority Data
Nov. 4, 2016 (KR) 10-2016-0146477

(51) Int. Cl. A61K 36/35; A61P 35/00; A23L 33/105

(52) U.S. Cl. CPC A61K 36/35; A23V 2002/00; A23L 33/105; A61P 35/00

(57) ABSTRACT

A composition for prevention or treatment of liver cancer includes a Nardostachys jatamansi extract. The composition exhibits anti-cancer effects on liver cancer and/or specific liver cancer resistant to antibiotics. The composition can be included, for example, in a pharmaceutical composition or a health functional food.

### TECHNICAL FIELD

The present invention relates to a pharmaceutical composition for prevention or treatment of liver cancer and a health functional food for the same.

### BACKGROUND ART

Cancer is a disease at the first leading cause of death in Korea, and according to the report of the national statistical office (NSO) in 2009, three (3) major causes of all deaths in Korea are cancer, cardiovascular disease and cerebrovascular disease, which occupy about 50% of all deaths. Therefore, cancer-caused death has become a serious problem in society. In addition, among OECD countries, Korea is the first in liver cancer incidence. Further, in 2011, 27.8% of all deaths was due to cancer, and among them, lung cancer was ranked first with 22.2% and liver cancer was ranked second with 15.3%. In recent years, despite of noticeably advanced treatment methods of liver cancer (liver resection, liver transplantation, chemotherapy, etc.), it was reported that five (5) years survival of the liver cancer is as low as third following pancreatic cancer and lung cancer, and shows a rise of 26.7% in terms of a change in relative survival rate, however, is classified as an incurable cancer due to 70% or more recurrence within five years after surgery.

In a case of early liver cancer, a surgical resection of liver, that is, hepatectomy is adopted as the best treatment, and in a case of the liver cancer (with three or less nodules of 3 cm or less), liver transplant is executed in an early stage, transarterial chemoembolization (TACE) is used in an intermediate stage, and the terminal liver cancer is subjected to systemic anticancer chemotherapy as a liver cancer treatment method, depending on stages of the disease.

If TACE is failed or there is vascular invasion, systemic anticancer chemotherapy has been employed till now and sorafenib (NEXABAR) only is used. It has been known that, when sorafenib orally administered as a multikinase inhibitor, this drug could extend the life only about three (3) months, compared to a placebo dose group. Further, major side effects such as diarrhea, fatigue, decrease in body weight, hand and foot syndrome, etc. have been reported. sorafenib exhibited partial response only in about 5% or less of 137 patients in Phase 2 clinical trials. Further, since sorafenib-resistant patients have been recently reported, an assistant drug capable of replacing sorafenib or suppressing sorafenib resistance in treatment of liver cancer, and/or a novel and effective drug useable for liver cancer in a variety of applications are urgently needed.

Meanwhile, Nardostachys jatamansi (NJ) has been broadly used in stomach pain, stomach cramps, gastrointestinal bloating, functional gastrointestinal disorder, vomiting, headache, beriberi, etc., applied widely as a tonic, stimulant or anti-epileptic drug, and further used to treat epilepsy, hysteria, palpitations or the like. However, it is not reported yet that Nardostachys jatamansi (NJ) has liver cancer therapeutic effects.

In studies of crude drugs including Nardostachys jatamansi, the inventors have firstly discovered that Nardostachys jatamansi extract has effects on liver cancer therapy, and the present invention has been completed on the basis of the finding.

### DISCLOSURE

### Technical Problem

Accordingly, it is an object of the present invention to provide a composition and a health functional food, each of which has liver cancer therapeutic and preventive effects.

### Technical Solution

1. A pharmaceutical composition for prevention or treatment of a liver cancer, comprising Nardostachys jatamansi extract.

2. The composition according to the above 1, wherein the liver cancer is a liver cancer resistant to sorafenib.

3. The composition according to the above 1, wherein the Nardostachys jatamansi extract is an extract in an organic solvent or a hot water.

4. The composition according to the above 1, wherein the Nardostachys jatamansi extract is an extract in ethanol.

5. A health functional food for prevention or treatment of liver cancer, comprising Nardostachys jatamansi extract.

6. The health functional food according to the above 5, wherein the liver cancer is a liver cancer resistant to sorafenib.

7. The health functional food according to the above 5, wherein the Nardostachys jatamansi extract is an extract in an organic solvent or a hot water.

8. The health functional food according to the above 5, wherein the Nardostachys jatamansi extract is an extract in ethanol.

### Advantageous Effects

The composition and the health functional food of the present invention, each of which includes Nardostachys jatamansi extract, may exhibit anti-cancer effects against liver cancer and/or specific liver cancer resistant to anti-cancer drugs.

### DESCRIPTION OF DRAWINGS

FIG. 1 is graphs illustrating a comparison of efficacies between natural extracts and sorafenib in Huh7 cells through MTT assay.

FIG. 2 is a graph illustrating efficacies of natural extracts in SRH cells through MTT assay.

FIGS. 3 and 4 are graphs illustrating effects of treatment of HCCLM3 with sorafenib.

FIG. 5 is graphs illustrating effects of treatment of HCCLM3 with Nardostachys jatamansi extract and Lophatheri Herba extract, respectively.

FIG. 6 is a graph illustrating effects of treatment of Huh7 cells with filtered Nardostachys jatamansi extract.

FIG. 7 is diagrams schematically illustrating animal modeling-1 for in vivo efficacy test.

FIGS. 8 to 10 are photographs illustrating results of hepatocellular carcinoma modeling-1 with administration of HCCLM3 to Balb/c nu/nu mouse.

FIG. 11 is a graph illustrating liver weight/body weight in the hepatocellular carcinoma modeling-1 with administration of HCCLM3 to Balb/c nu/nu mouse.

FIG. 12 is graphs illustrating analyzed results of serum in the hepatocellular carcinoma modeling-1 with administration of HCCLM3 to Balb/c nu/nu mouse.

FIG. 13 is graphs illustrating effects of treatment of FL83b cell line with Nardostachys jatamansi extract.

FIG. 14 is diagrams schematically illustrating animal modeling-2 for in vivo efficacy test.

FIGS. 15 and 16 are photographs illustrating results of hepatocellular carcinoma modeling with administration of Hepa1-6 to C57/BL6 mouse.

FIG. 17 is a graph illustrating liver weight/body weight in the hepatocellular carcinoma modeling with administration of Hepa1-6 to C57/BL6 mouse.

FIG. 18 is diagrams illustrating a change in signaling pathway after treatment using Nardostachys jatamansi extract through western blot.

FIG. 19 is a schematic view illustrating ERK activity inhibition by treatment using Nardostachys jatamansi extract.

### BEST MODE

The present invention discloses a pharmaceutical composition for prevention or treatment of liver cancer and a health functional food for the same, and more particularly, a pharmaceutical composition and a health functional food, both of which can prevent or treat liver cancer due to including Nardostachys jatamansi extract, so as to exhibit anti-cancer effects on liver cancer and/or specific liver cancer resistant to antibiotics.

Hereinafter, the present invention will be described in detail.

The pharmaceutical composition for prevention or treatment of liver cancer according to the present invention includes Nardostachys jatamansi extract.

The term “extract” refers to an active ingredient isolated from natural substance. The extract may be obtained by extraction using water, an organic solvent or a mixed solvent thereof, and may include liquid extract and dry powders or all formulations thereof. Further, a product obtained by suspending the extract material after the extraction with water or an organic solvent, followed by fractionation using hexane, chloroform, butanol, ethyl acetate, etc. may also be included within the scope of the extract of the present invention.

The term “prevention” means all actions to suppress or delay onset of liver cancer by administration of a composition and/or a health functional food.

The term “treatment” or “improvement” means all actions to improve or beneficially change symptoms of metabolic disorder by administration of the composition and/or the health functional food according to the present invention. Persons of ordinary skill in the art to which the present invention pertains (‘those skilled in the art’) will understand the exact criteria for diseases, to which the present inventive composition may be effective, with reference to data presented by the Korean Medical Association or the like, and will be able to determine the degree of improvement, enhancement and treatment.

The liver cancer may include, for example, hepatocellular carcinoma, cholangiocarcinoma, vascular sarcoma, etc., and further include metastasis occurring in organs other than the liver and spreading to the liver even if it was not generated in the liver.

The liver cancer may be a liver cancer having resistance to an anticancer agent, and preferably, a liver cancer resistant to sorafenib.

In addition, the liver cancer may be a progressive liver cancer, and the composition of the present invention can be applied thereto regardless of the progressing stages.

Nardostachys jatamansi (NJ) refers to root stocks and/or roots of perennial Nardostachys chinensis Batalin or the same genus plants and may be called spikeNard, petty morrel (i.e., gomichi), life-of-man (i.e., hyangsong in Korea), etc.

Nardostachys jatamansi extract may be extracted from any region of Nardostachys jatamansi, and preferably from the roots of Nardostachys jatamansi. Further, the liquid extract may be obtained by extraction using water or an organic solvent. In this case, the organic solvent may include, for example, lower alcohol, acetone, chloroform, methylene chloride, ether, ethyl acetate, hexane and the like. The lower alcohol may include, for example, methanol, ethanol, propanol and butanol, and preferably, ethanol.

When using water, dried Nardostachys jatamansi or Nardostachys jatamansi powders are diluted by adding 1 to 20 times, preferably 5 to 15 times, and more preferably, 10 times water, and may be subjected to extraction at a temperature of 80 to 100° C. for 1 to 24 hours, preferably, 2 to 6 hours, and more preferably, 2 hours and filtration, thereby preparing Nardostachys jatamansi root extract in hot water. For the organic solvent-based extract, an organic solvent of 1 to 20 times, preferably 5 to 15 times, and more preferably, 10 times the amount of Nardostachys jatamansi is added and the mixture may be subjected to extraction at room temperature (20 to 30° C.) or in a warm condition for 10 to 100 hours, preferably, 15 to 40 hours, and more preferably, for 24 hours, followed by filtration and concentration under reduced pressure, thereby preparing Nardostachys jatamansi extract in the organic solvent. In the above extraction processes, if necessary, the extracting step may be repeated two or more times, and the resultant extract may be produced in a powder form by freeze-drying, spray drying or drying under reduced pressure.

The composition of the present invention may further include a pharmaceutically acceptable carrier. The pharmaceutically acceptable carrier means a pharmaceutically acceptable substance, composition or vehicle such as liquid or solid fillers, diluents, excipients or solvents, each of which serves to transport active ingredients from one organ or a part of the body into other organs or other parts. The composition of the present invention may be prepared as a medicament by adding one or more pharmaceutically acceptable carriers as well as the Nardostachys jatamansi extract. Herein, the “pharmaceutically acceptable” means being physiologically acceptable and generally means that an allergic reaction or similar reaction is not caused in the administration to humans. Such carriers may include, for example, saline, buffered saline, water, glycerol, ethanol, and the like, without particular limitation thereof, and any suitable formulation known in the art may be used.

Agents for pharmaceutical preparation of the inventive composition may be administered orally at the time of clinical administration, and may be used in general forms of pharmaceutical formulation. The formulation may be prepared using fillers, extenders, binders, wetting agents, disintegrating agents, diluents such as surfactants, or excipients, which are generally used in the art. A solid formulation for oral administration may include, for example, tablets, pills, discutients, granules, capsules, etc. Further, a liquid formulation for oral administration may include, for example, suspensions, oral solutions, emulsions, syrups, etc. Other than simple diluents commonly used in the art such as water, liquid and paraffin, various excipients, for example, wetting agents, sweeteners, aromatics and preservatives, may be further included.

Further, herbal medicinal agents that can be added to the composition of the present invention may be any pharmaceutically acceptable herbal medicinal material, and may include, for example, Angelicae tenuissimae radix, Gastrodiae phizoma, Bapleuri radix, Angelicae gigantis radix, Persicae semen, Cinnamomi romulus, Rhei rhizoma, Glycyrrhizae radix (licorice), Cnidii rhizoma, Aurantii nobilis pericarpium, Alismatis rhizoma, Coptidis rhizoma, Scutellariae radix, Hoelen, Paeoniae radix (Peony), Atractylodis rhizoma alba, Phellodendri cortex, Gardeniae fructus (gardenia), Pinelliae tuber, Uncaria ramuluset uncus, Ponciri fructus, Gingseng, Liriopis tuber, Polygalae radix, Acori graminei rhizome, Atractylodis rhizoma alba, Chrysanthemi flos, Ledebouriellae radix, Zingiberis rhizoma crudus (ginger), Natrii sulfas, Zizyphi fructus, Salviae radix, Mautan radicis cortex, Rehmanniae radix, Menthae herba (mint), Dioscoreae rhizoma, Polyporus, Polygonimultiflori radix, Allii tuberosi semen, Cassiae semen, Lycii fructus, Araliae cordatae radix, Eucommiae cortex, Hedyotis herba, Saururus herba, Artemisiaecapillaris herba, Anemarrhenae rhizoma, Carthami flos (Safflower), Astragali radix, Lycopodium, Ginkgonis folium (gingko leaf), Polygonati rhizoma, Nelumbinis semen, Fossilia ossis mastodi, Lycii radicis cortex, Achyranthis radix, Rehmanniae radix preparata, Perilae semen (black sesame), Thujae semen, Hordei fructus germinates (malt), Cuscutae semen, Morindae radix, Pini koraiensis radix (black pine), which are used alone or in combination thereof.

The composition of the present invention may be administered orally or parenterally.

In the present disclosure, the term “administration” means introduction of the pharmaceutical composition of the present invention to a subject by any suitable method.

The parenteral administration may include intravenous, intramuscular, intra-arterial, intramedullary, intrathecal, intracardiac, transdermal, subcutaneous, intraperitoneal, intranasal, secretary, topical, sublingual or rectal administration, but it is not limited thereto.

In a case of a formulation for oral administration, the composition of the present invention may be prepared in any form of powders, granules, tablets, pills, sugar tablets, capsules, liquids, gels, syrups, slurries, suspensions, etc. by any method known in the art. For instance, the oral formulation may be prepared in a form of tablets or sugar tablets by blending an active ingredient with a solid excipient, pulverizing the mixture, adding a proper adjuvant thereto and processing the same in a granular mixture. The proper excipient may include, for example, sugars such as lactose, dextrose, sucrose, sorbitol, mannitol, xylitol, erythritol and maltitol, starches such as corn starch, wheat starch, rice starch and potato starch, celluloses such as cellulose, methyl cellulose, sodium carboxymethyl cellulose and hydroxypropylmethyl-cellulose, fillers such as gelatin, polyvinylpyrrolidone, etc. Further, occasionally, cross-linked polyvinyl pyrrolidone, agar, alginic acid or sodium alginate, etc. may be added as a disintegrating agent. Moreover, the composition of the present invention may further include anti-coagulants, lubricants, wetting agents, flavoring agents, emulsifying agents and/or preservatives.

In a case of a formulation for parenteral administration, the formulation may be obtained in a form of injections, creams, lotions, external ointments, oils, moisturizers, gels, aerosols and/or nasal inhalers by any method known in the art.

A suitable total daily dose of the formulation according the present invention may be determined by a medical attendant within a proper medical determination scope. A concrete and therapeutically effective amount to a specific subject is preferably and differently determined depending upon, for example, desired reaction type and degree thereof, age, body weight, general health condition, sex and diet of the subject, whether alternative formulations are applied or not, details of the composition to be used, administration time, administration route, secretion rate of the composition, treatment duration, other drugs used along with or simultaneously used with the specific composition, a variety of factors and similar factors well known in the medical field. Therefore, the preferred dosage of the composition according to the present invention may be determined in consideration of the foregoing conditions and may be suitably selected by those skilled in the art. However, in order to attain preferred effects, the composition of the present invention may be administered in an amount of 0.0001 to 500 mg/kg, preferably, 50 to 300 mg/kg, more preferably, 100 to 200 mg/kg, and most preferably, 100 mg/kg, to a dry weight of the Nardostachys jatamansi extract, which can be administered once a day or in several-divided does a day, but it is not limited thereto.

The composition of the present invention may also include the formulation in dosage unit. The formulation may exist in discrete dosage forms, for example, tablets, coated tablets, capsules, pills, suppositories and ampoules, and a content of effective compound in a medicament may correspond to the fraction or multiples of a single dose. Dosage unit may include, foe example, 1, 2, 3 or 4 times or ½, ⅓ or ¼ times of the discrete dose. The discrete dose preferably contains an amount of effective compound administered at one time, which corresponds to all, ½, ⅓ or ¼ times the usual daily dose.

If necessary, the composition of the present invention may further include Lophatheri Herba extract.

The Lophatheri Herba extract induces apoptosis of liver cancer cells depending on a mechanism different from that of the Nardostachys jatamansi extract, and therefore, the composition of the present invention may further include the Lophatheri Herba extract, thereby improving prevention or treatment effects of liver cancer.

For prevention or treatment of liver cancer, the composition of the present invention may be used alone or in combination with any of conventional methods such as surgery, hormone therapy, chemotherapy and/or use or biological response modifiers.

A subject who is able to accept application of the composition of the present invention may include any animal possibly taken with such disease as described above, and may include not only human and primates, but also other animals including, for example, livestock such as cattle, pigs, sheep, horses, dogs and cats.

Further, the present invention may provide a health functional food for prevention or improvement of liver cancer, which includes Nardostachys jatamansi extract.

According to one embodiment of the present invention, the health functional food of the present invention may further include Lophatheri Herba extract.

The health functional food of the present invention may include beverages (alcohol-containing beverages), fruits and processed foods thereof (e.g., canned fruit, bottled fruit, jams, marmalade, etc.), fishes, meats and processed foods thereof (e.g., ham, sausage corn beef, etc.), breads and noodles (e.g., thick noodles, soba, ramen, spaghetti, macaroni etc.), fruit juice, various drinks, cookies, taffy, dairy products (e.g., butter, cheese, etc.), edible vegetable oils and fats, margarine, plant proteins, retort foods, frozen foods, various seasonings (e.g., soybean paste, soy sauce, sauce, etc.) or the like, which include the above extract.

Further, the health functional food of the present invention may be formulated in a form of tablets, pills, discutients, granules, powders, capsules, liquid dosage forms, etc. These may be formulated while further including one or more of carriers, diluents, excipients and additives.

Further, the additives possibly included in the health functional food of the present invention may include one or more components selected from the group consisting of natural carbohydrates, spices, nutrients, vitamins, minerals (electrolytes), flavors (synthetic flavoring agents and natural flavoring agents, etc.), coloring agents, fillers (cheese, chocolate etc.), pectic acid and its salts, alginic acid and its salts, organic acid, protective colloidal thickeners, pH adjusting agents, stabilizing agents, preservatives, antioxidants, glycerin, alcohol, carbonating agents and fruit pulp.

Examples of the natural carbohydrates may include: monosaccharide, e.g., glucose, fructose, etc.; disaccharides, e.g., maltose, sucrose, etc.; and polysaccharides, e.g., conventional sugars such as dextrin, cyclodextrin, etc. and sugar alcohol such as xylitol, sorbitol, erythritol, etc. The flavors advantageously used herein may include natural flavors (thaumatin, stevia extract (e.g., rebaudioside A, glycyrrhizin, etc.) and synthetic flavors (saccharin, aspartame, etc.).

In addition to the above substances, the health functional food of the present invention may further include various nutrients, vitamins, minerals (electrolytes), flavors such as synthetic flavors and natural flavors, coloring agents and fillers (cheese, chocolate, etc.), pectic acid and its salts, alginic acid and its salts, organic acid, protective colloidal thickeners, pH adjusting agents, stabilizers, preservatives, glycerin, alcohol, carbonating agents used in soft drinks, or the like. Moreover, the health functional food of the present invention may include fruit pulp for production of natural fruit- and vegetable-based juice drinks. These components may be used independently or in combination.

Specific examples of the carriers, excipients, diluents and additives may include lactose, dextrose, sucrose, sorbitol, mannitol, erythritol, starch, gum acacia, calcium phosphate, alginate, gelatin, calcium phosphate, calcium silicate, macrocrystalline cellulose, polyvinyl pyrrolidone, cellulose, methyl cellulose, water, sugar syrup, methyl hydroxybenzoate, propyl hydroxybenzoate, talc, magnesium stearate, mineral oil and mixtures thereof, etc., but it is not limited thereto.

A content of the health functional food of the present invention as an active ingredient in the aforementioned formulation may be adequately adjusted according to use forms and purposes, user's conditions, type and severity of symptoms, and may range from 0.001 to 99.9% by weight (‘wt. %’), preferably, 0.01 to 50 wt. % in terms of solid content by weight, but it is not limited thereto.

Hereinafter, the present invention will be described in detail to illustrate the present invention by way of following examples.

### EXAMPLE

In the following examples and drawings, WSY-0 indicates Ulmi cortex extract, WSY-1 indicates Nardostachys jatamansi extract, WSY-3 indicates Lophatheri Herba extract and WSY-37 indicates Torilis japonica extract.

Preparation of Ulmi cortex extract (WSY-0), Nardostachys jatamansi extract (WSY-1), Lophatheri Herba extract (WSY-3) and Torilis japonica extract (WSY-37)

Ulmi cortex, Nardostachys jatamansi, Lophatheri Herba and Torilis japonica purchased from a typical manufacturer (Omni Herb, Seoul, Korea) were dried in the shade at room temperature for 5 days and then pulverized. The pulverized Ulmi cortex, Nardostachys jatamansi, Lophatheri Herba and Torilis japonica were each subjected to hydrothermal extraction with distilled water of 10 times the weight of raw material for about 2 hours.

Further, the pulverized Ulmi cortex, Nardostachys jatamansi, Lophatheri Herba and Torilis japonica were each also subjected to extraction with ethanol of 10 times the weight of raw material for 24 hours.

MTT Assay

Hepatoma cell proliferation inhibitory efficacies of the Ulmi cortex extract (WSY-0), Nardostachys jatamansi extract (WSY-1), Lophatheri Herba extract (WSY-3) and Torilis japonica extract (WSY-37) in target cells or mice treated with the same were validated by MTT assay and effective concentrations thereof were determined.

Annexin V & PI Staining

In order to verify whether a hepatoma cell line proliferation suppressing pathway is an apoptosis process or a necrosis process due to cytotoxicity in treatment using the Ulmi cortex extract (WSY-0), Nardostachys jatamansi extract (WSY-1), Lophatheri Herba extract (WSY-3) and Torilis japonica extract (WSY-37), respectively, Annexin V & PI staining were implemented.

Establishment of Hepatocellular Carcinoma (HCC) in Small Animal Models

In order to induce HCC in a small animal model, a method of injecting chemicals such as DEN or a method of directly injecting HCC cells into the model is used. Although DEN treatment is convenient in view of experiment, this process entails a disadvantage in that a long period of time (about 50 weeks) is required for generation of tumors (‘tumorigenesis’). Therefore, in order to efficiently verify in vivo anticancer effects of natural extracts for a short period of time, an orthotopic implantation model of directly injecting HCC cells into the liver of a mouse was implemented.

The orthotopic implantation model is a method of inducing formation of cancer in liver tissues by directly injecting HCC cell lines such as Huh7 or HCCLM3 into hepatic portal vein or mesenchyma (liver lobe) of the mouse, and takes about 8 weeks to form tumors, which is considerably shorter than DEN, thus being properly used.

Efficacies of the Ulmi cortex extract (WSY-0), Nardostachys jatamansi extract (WSY-1), Lophatheri Herba extract (WSY-3) or Torilis Japonica extract (WSY-37) on HCC formation and suppression thereof have been validated through liver gross, H & E staining and serum analysis.

Study of Mechanism of Natural Extracts

For realization of anti-cancer effects and a mechanism of natural extracts, expression and phosphorylation of proteins involved in a representative signaling pathway in regard to tumorigenesis were verified through Western blot.

Results and Discussion

Induction and Verification of Apoptosis/Necrosis of Selected Natural Extracts

In vitro liver cancer growth inhibitory abilities of four (4) natural products (WSY-0, WSY-1, WSY-3, and WSY-37) were verified by the MTT assay. Results thereof are shown below.

With respect to the 4 types of natural products, total 8 types of extracts were prepared through hydrothermal extraction and ethanol extraction, respectively. These extracts were identified to have in vitro liver cancer growth inhibitory ability in 5 types of liver cancer cell lines (Huh7, HepG2, Hep3B, PLC/PRF5, and SK-hep1).

From the results, it was observed that the materials obtained through hydrothermal extraction exhibit higher in vitro liver cancer growth inhibitory ability than those obtained through ethanol extraction. Further, in treatment using each of three ethanol extracts in an amount of 200 μg/mL except WSY-37, the cell growth inhibitory ability was identified in 4 types of liver cancer cell lines except Hep G2 cells.

Table 1 showed the comparison of anti-cancer effects in the treatment using Ulmi cortex extract (A), Nardostachys jatamansi extract (B) and Lophatheri Herba extract (C) in each amount of 200 μq/mL.

TABLE 1

| | HepG2 | Huh-7 | Hep3B | PLC/PRF5 | SK-hep1 |
|---|---|---|---|---|---|
| A: WSY-0 | — | 37% | 40% | 40% | 38% |
| B: WSY-1 | — | 62% | 52% | 42% | 58% |
| C: WSY-3 | — | 50% | 50% | 50% | 37% |

Based on the results of the preceding findings above, with respect to 3 types of extracts in ethanol (WSY-0, WSY-1 and WSY-3) other than WSY-37, whether apoptosis/necrosis was induced or not has been identified through Annexin V/PI staining.

When treating with WSY-0, HCC observed through Annexin V/PI staining did not show induction of cell death.

When treating with WSY-1, after treatment for 24 hours, it was identified that 50% or more cell death was induced using 100 μg/mL of WSY-1, and 90% or more cell death was induced using 200 μg/mL of WSY-1, respectively. Further, 50% or more cell death was observed as apoptosis.

When treating with WSY-3, it was observed that necrosis was induced in a concentration-dependent manner, and in particular, necrosis was induced in 90% or more of cells treated with 200 μg/mL of WSY-3.

Consequently, it was found that WSY-1 extract in ethanol and WSY-3 extract in ethanol could induce cell death, wherein WSY-1 exhibited apoptosis-dependent cytotoxicity whereas WSY-3 had necrosis-dependent cytotoxicity.

Comparison of Efficacies between Natural Extracts and Sorafenib

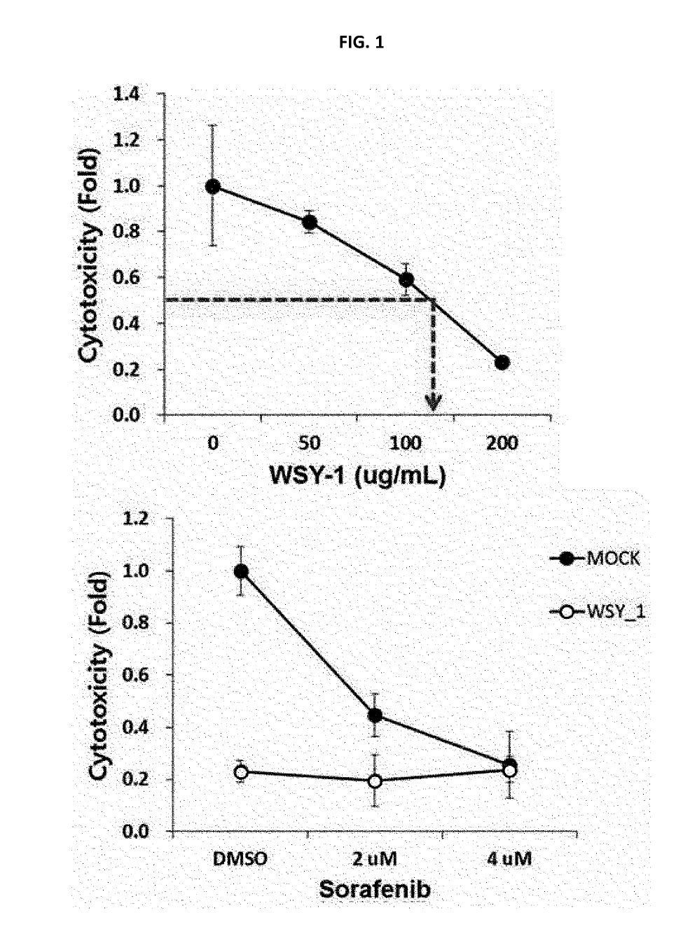

When treating Huh7 cells with sorafenib for 48 hours, it was identified that about 60% cell death was induced with 2 μM of sorafenib and about 80% cell death was induced with 4 μM of sorefenib, respectively (see FIG. 1).

When treating with 200 μg/mL of WSY-1, it was found that about 80% cell death was induced, which was similar to the effects of the treatment with 4 μM sorafenib.

According to the present experimental results, it could be seen that 200 μg/mL WSY-1 may induce cell death of hepatocellular carcinoma (HCC) in Huh7 cells in a similar level as 4 μM sorafenib.

Verification of Effects of Natural Extracts in Sorafenib-Resistant Huh7 Cell Line (SRH)

In the Huh7 cell line-derived sorafenib-resistant cell line (SRH), efficacy of WSY-1 was subjected to validation.

Treatment using 0 μg/mL, 50 μg/mL, 100 μg/mL and 200 μg/mL of each natural extract was conducted, and after 48 hours, MTT assay was implemented.

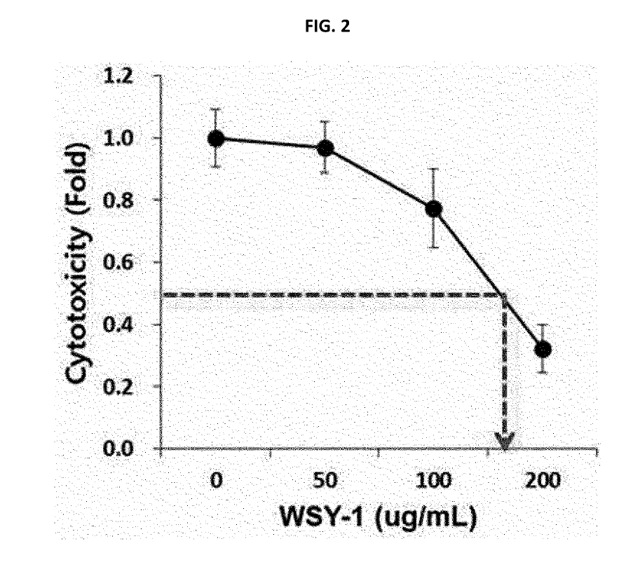

As compared to the treatment of Huh7 cells, anti-cancer effects were slightly reduced, however, it was identified that at least 50% cell death was induced in the cells treated using 200 μg/mL of natural extract (see FIG. 2).

Verification of Effects of Natural Extracts in Hyper-Metastatic HCC Cell Line (HCCLM3)

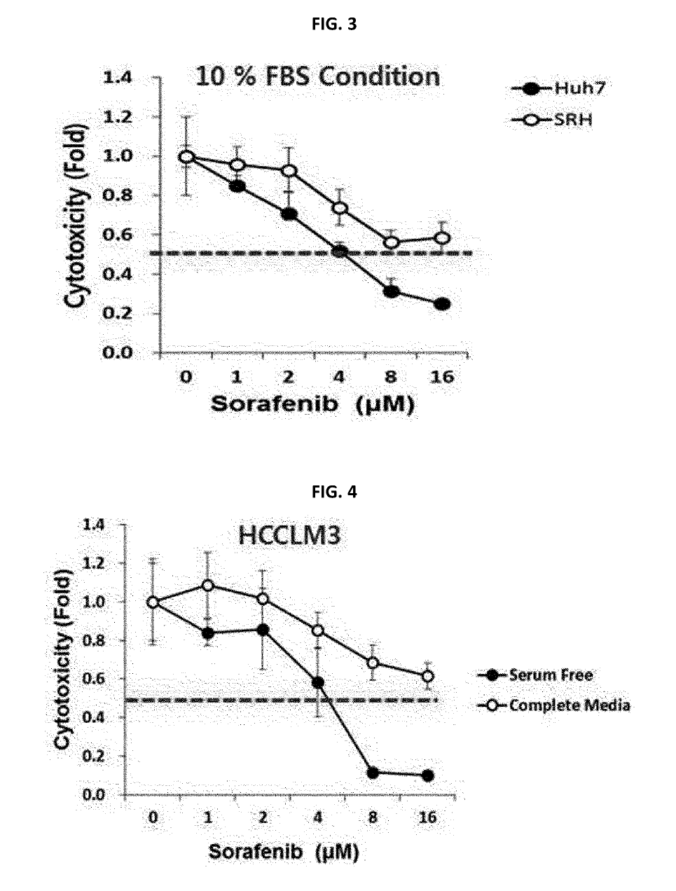

HCCLM3 cells showed 50% or less apoptotic effects even after treatment using 16 μM sorafenib for 48 hours similar to sorafenib-resistant cell line (SRH) (see FIGS. 3 and 4).

By treatment of HCCLM3 cells using WSY-1 or WSY-3 extract in ethanol to identify cell death effects, in vitro anti-cancer effects of the natural extracts on cell lines having different sorafenib sensitivities were investigated.

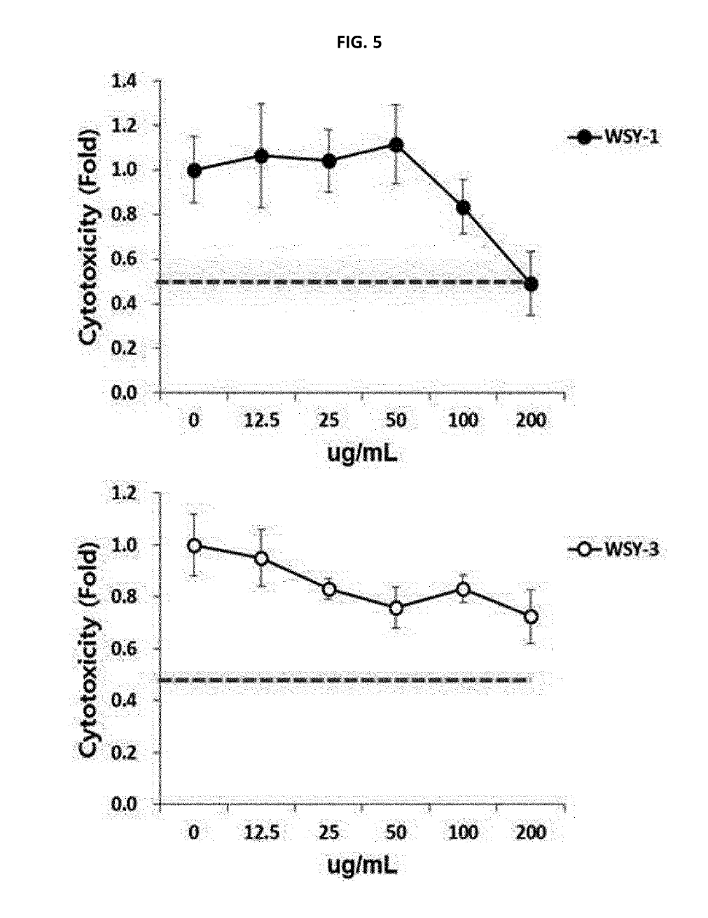

When treating HCCLM3 cells with WSY-1 or WSY-3, cell death effects by WSY-3 at a concentration of 200 μg/mL were not observed. On the other hand, in a case of WSY-1 treatment, about 50% cell death effect was observed (see. FIG. 5).

According to the present experimental results, it was identified that WSY-1 had in vitro anti-cancer efficacy in not only sorafenib-resistant Huh7 cells but also hyper-metastatic HCC cell line, that is, HCCLM3.

Based on the above experimental results, it could be understood that WSY-1 among the above 4 types of natural extracts had the most excellent in vitro anti-cancer effects, and therefore, WSY-1 was selected as a subject for in vivo efficacy experiment.

Validation of Primary In Vivo Efficacy Using Balb/c Nude Nude Mouse (Balb/c nu/nu Mouse) and HCCLM3 Cell Line

In order to verify in vivo anti-cancer effects of natural extracts on HCC, an optimum Hepatocellular carcinoma (HCC) small animal model was established, and an orthotopic implantation method of directly injecting HCC into the liver of a mouse was adopted.

The HCC was induced by directly injecting 2×10⁶ HCCLM3 cells into the liver of Balb/c nude nude mouse through the liver portal vein, and then, WSY-1 or sorafenib was administered so as to verify anti-cancer effects.

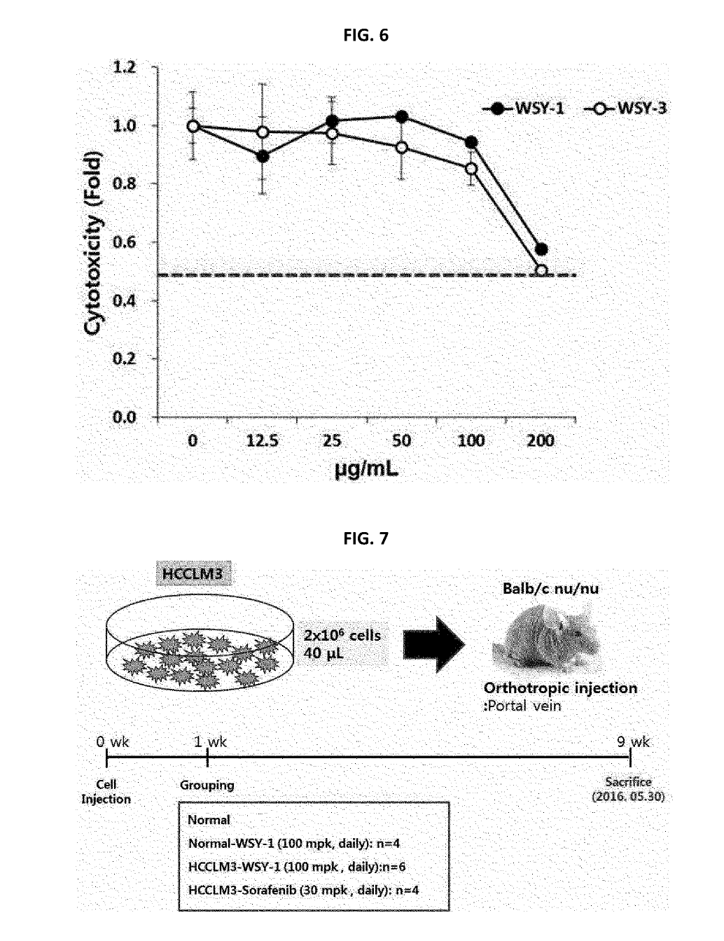

Prior to the verification of in vivo anti-cancer effects, in order to determine an administration route of the natural extract, each of WSY-1 and WSY-3 dissolved in distilled water was subjected to filtration using 0.4 μm filter and apoptotic effects were re-examined. In a case of WSY-1, it showed that the apoptosis effect on liver cancer cells was decreased after filtration, compared to before filtration (see FIG. 6).

That is, it is considered that anti-cancer effects of administrated WSY-1 would be decreased if WSY-1 undergoes a filtration step. Therefore, in order to administer WSY-1 to an animal without filtration after dissolving the same in a biocompatible solution, this extract would be administered via an oral administration route.

Based on the existing research papers for natural extracts, administration dose and frequency of WSY-1 were determined to be daily 100 mpk (1 mg per kg body weight).

Based on the above description, experiments for validation of in vivo anti-cancer efficiency of WSY-1 were implemented.

To each of balb/c nude nude mice with 6 weeks of age, 2×10⁶ HCCLM3 cells were directly injected into the liver through the liver portal vein. After 1 week, the mice with induced HCC were randomly divided into groups, followed by administration of WSY-1 and sorafenib. 100 mpk of WSY-1 and 30 mpk of sorafenib were daily given by oral administration for 8 weeks, respectively (see FIG. 7).

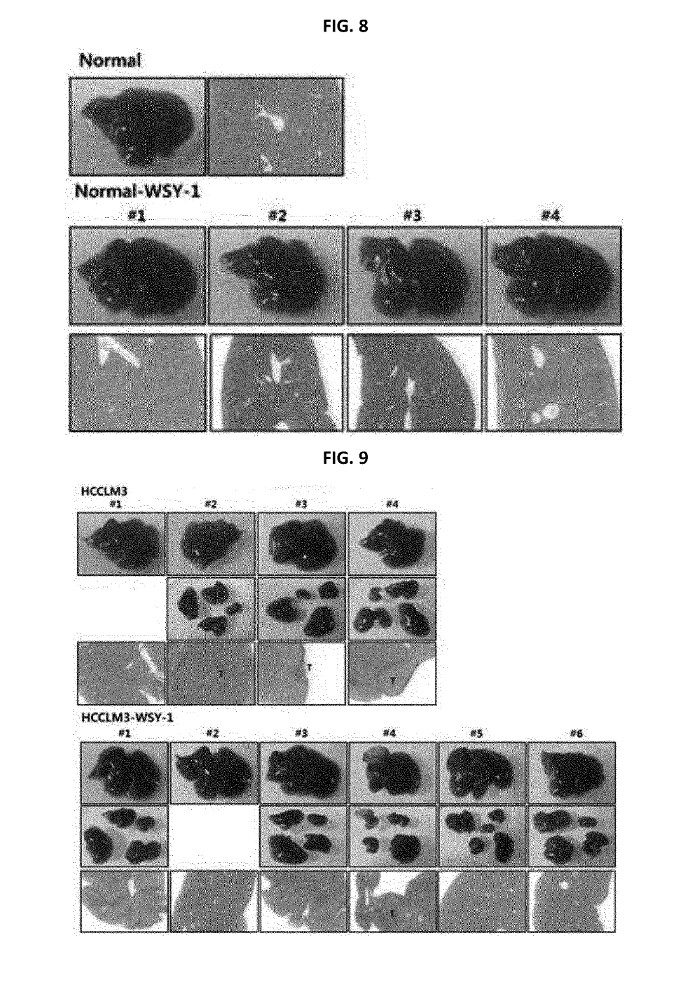

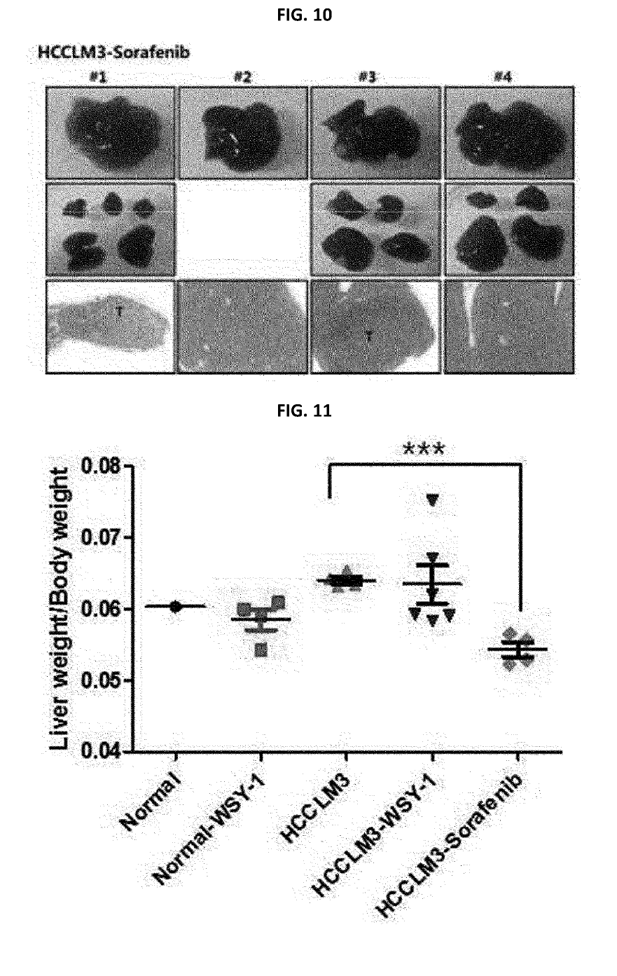

In order to identify in vivo toxicity of WSY-1, mice without HCCLM3 administration were used as a negative control while an experimental group was set to include normal mice subjected to administration of 100 mpk WSY-1 daily. After 8 weeks, the mice were sacrificed to analyze anti-cancer effects (see FIGS. 8 to 10).

As a result of analyzing liver weight/body weight in the same model, it was found that the liver weight/body weight value was decreased in the mice with administration of HCCLM3 and then sorafenib, compared to the mice with administration of HCCLM3 only (see FIG. 11).

With respect to the same model, levels of alanine aminotransferase (ALT) and aspartate aminotransferase (AST) in the serum were analyzed.

AST is better known as a glutamic oxalacetic transaminase (GOT). This is an enzyme existing in the heart, kidney, brain, muscle, etc. other than liver cells, and when such cells are damaged, AST concentration is increased. On the other hand, ALT is better known as a glutamic pyruvate transaminase (GPT). This is an enzyme mostly existing in the liver cells, and when the liver cells are damaged, ALT concentration is increased.

Each of the ALT and AST levels means a value on a blood test that can determine whether liver functions are degraded or not.

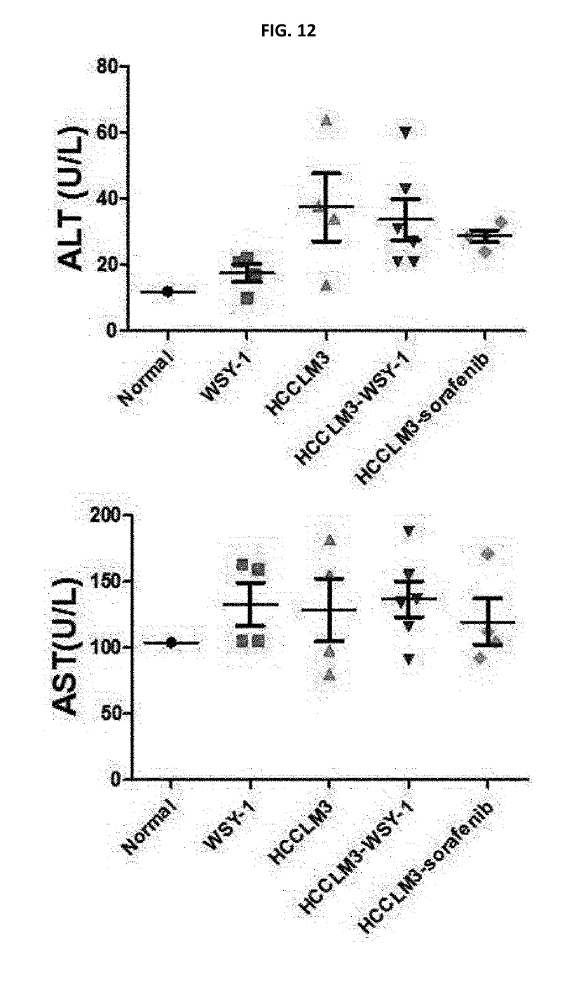

Serum ALT levels in the mice receiving daily administration of 100 mpk WSY-1 did not show any significant difference compared to the same levels in normal mice. From these results, oral administration of WSY-1 is not concerned to cause harmful effects on liver functions (see FIG. 12).

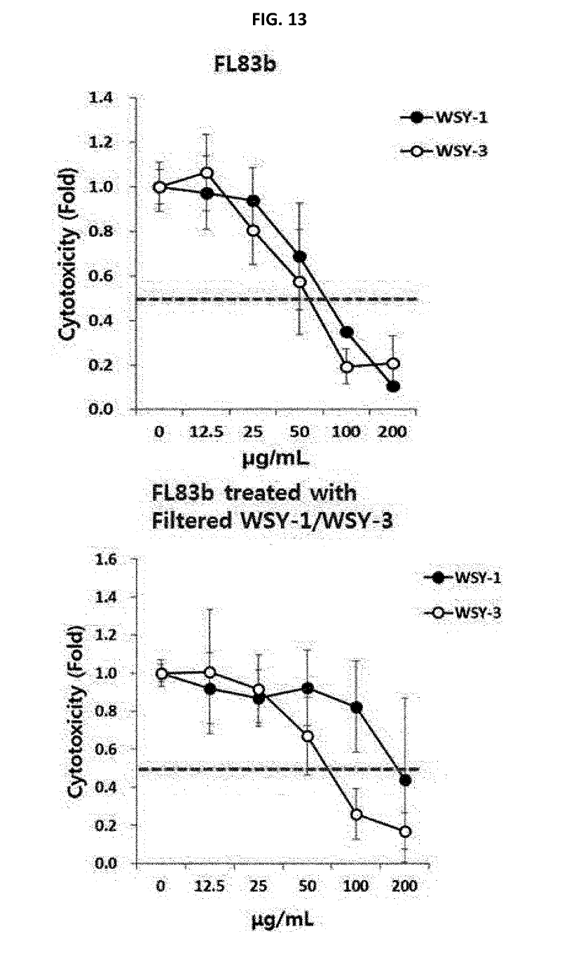

After treatment of FL83b as hepatocyte cell lines in normal mice using WSY-1 and WSY-3, respectively, MTT assay was implemented. As a result of the assay, cell death induction was identified (see FIG. 13).

Accordingly, in vivo toxicity of WSY-1 was concerned. However, as compared to the normal mice, serum ALT and AST levels in the mice receiving oral administration of 100 mpk WSY-1 daily for 8 weeks were not significantly varied. From these results, it is predicted that the corresponding administration dosage and frequency would not cause toxicity to the liver.

As a result of the primary in vivo anti-cancer efficacy experiment, it is predicted that WSY-1 does not cause toxicity to the liver if 100 mpk WSY-1 is daily given by oral administration. Based on the results of the present experiment, a secondary in vivo anti-cancer efficacy experiment was implemented.

Validation of Secondary In Vivo Efficacy Using C57/BL6 and Hepa1-6 Cell Line

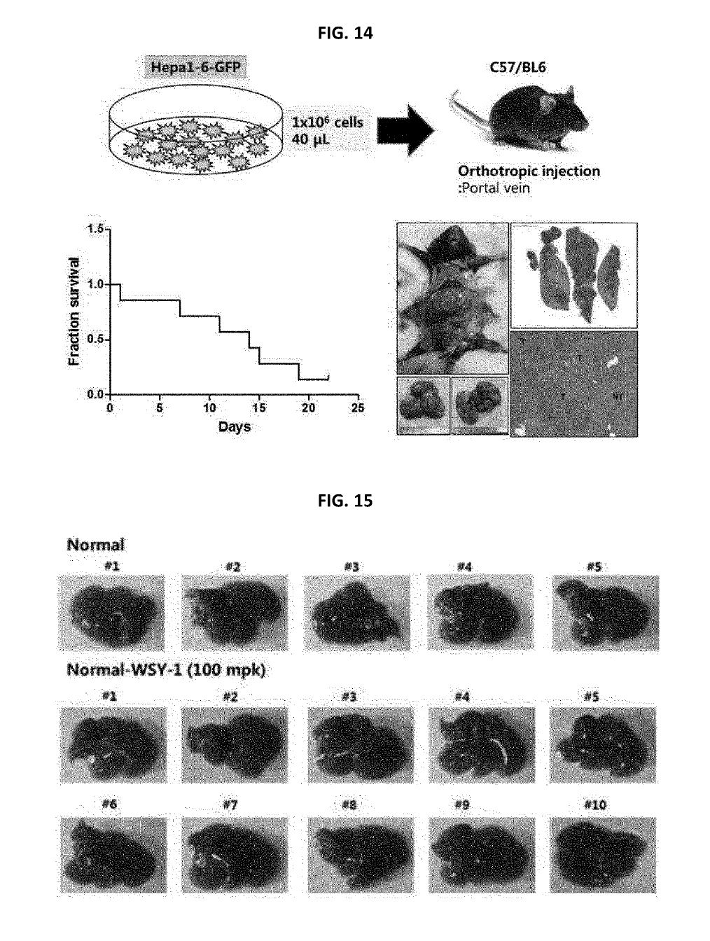

In order to establish an optimal model for validation of in vivo efficacy of WSY-1, a variety of modeling methods has been attempted. Among those, when Hepa1-5 cell line was administered to C57/BL6 mouse through the liver portal vein, efficient formation of HCC was identified. When administering 1×10⁶ cells, it was observed that most of the mice showed mortality due to tumor formation after 3 weeks (see FIG. 14).

In the present study, based on results of the primary in vivo efficacy validation experiment and preliminary experiment, the following experiment conditions were set up and the secondary validation experiment was implemented.

Mouse Species: C57/BL6

Administered cell/administration route: 5×10⁵ Hepa1-6-GFP cells/liver portal vein

Dose/administration route/dosing frequency: WSY-1 100 mpk, sorafenib 30 mpk/oral administration/daily

5×10⁵ Hepa1-6GFP cells were administered to the liver portal vein of each 6-week-old C57/BL6 mouse. 3 days later, the mice were randomly grouped, followed by administration of WSY-1 and sorafenib for 3 weeks.

Numbers (n) of animals in the experimental group are as follows:

Normal mice: n=5

Normal mouse+100 mpk WSY-1: n=10

Hepa1-6+mock+: n=13

Hepa1-6+100 mpk WSY-1: n=13

Hepa1-6+30 mpk sorafenib: n=13

During 3 weeks administration period, death (mortality) due to tumorigenesis was induced in the following experimental groups. After termination of the administration, survived mice were sacrificed and analyzed for anti-cancer effects.

Hepa1-6+mock: 5 out of 13 mice were died

Hepa1-6+100 mpk WSY-1: 6 out of 13 mice were died

Hepa1-6+30 mpk sorafenib: 4 out of 13 mice were died

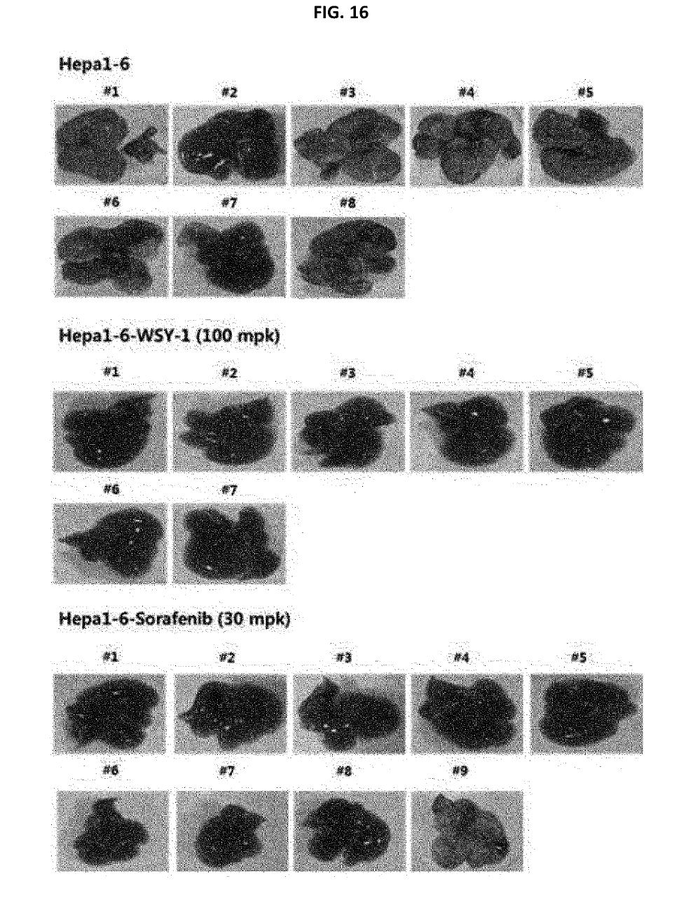

After termination of the administration, visual opinion to the excised liver gross is as follows (see FIGS. 15 and 16).

As compared to the livers of the normal mice, specific difference in the experimental group including mice with WSY-1 administration was not observed.

For Hepa1-6+mock experimental group, it was observed that the tumor was formed in all of 8 mice. Among those, 6 mice exhibited excessive tumorigenesis.

For Hepa1-6+100 mpk WSY-1 experimental group, tumor capable of being visually confirmed was not observed in the survived 7 mice.

For Hepa1-6+30 mpk sorafenib experimental group, tumor capable of being visually confirmed was observed in only 2 out of the survived 9 mice while the remaining 7 animals did not show the tumor.

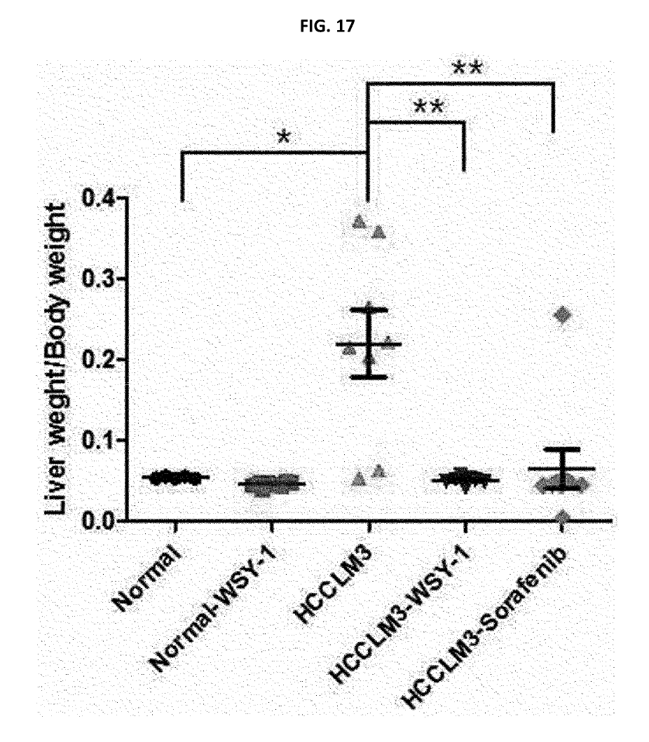

As a result of analyzing the liver weight/body weight in the same model, the levels were significantly increased in the mice injected with Hepa1-6 cells, compared to the normal mice. In contrast, it was identified that the levels were significantly decreased in the mice injected with Hepa1-6 cells and then with WSY-1 or sorafenib, compared to the mice injected with Hepa1-6 cells only (see FIG. 17).

Through liver gross observation and liver weight/body weight analysis, it was observed that the mice receiving administration of WSY-1 exhibited suppressed tumorigenesis to a level comparable with the mice receiving administration of sorafenib as the only target therapeutic agent till now.

Based on the present experimental results, it could be determined that WSY-1 administration in a HCC model might inhibit HCC formation, and further it could be predicted that the corresponding active ingredients were contained in WSY-1 extract in ethanol.

Analysis of Anticancer Mechanism of WSY-1

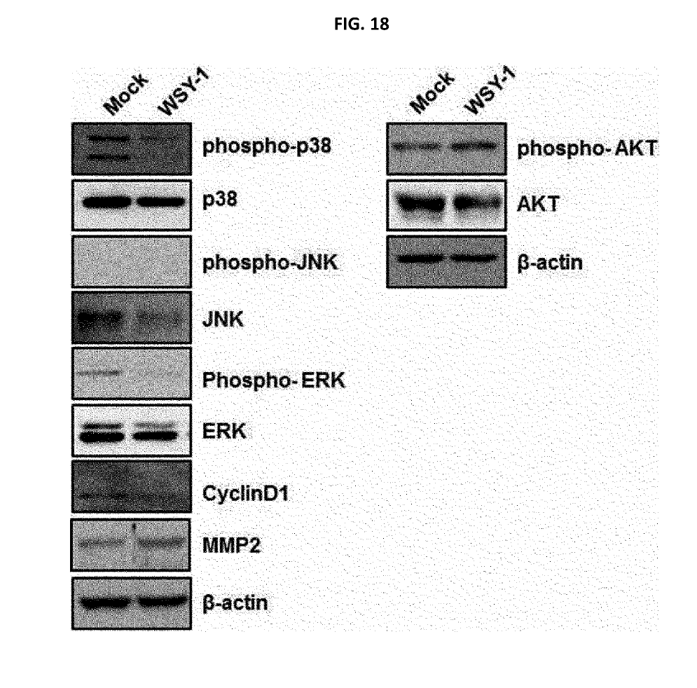

In order to analyze a mechanism in relation to anticancer effects of WSY-1, western blot was implemented. After treatment of Huh7 cells with WSY-1 at a concentration of 100 ug/mL for 24 hours, expression levels of proteins and whether there is phosphorylation of proteins or not were observed (see FIG. 18).

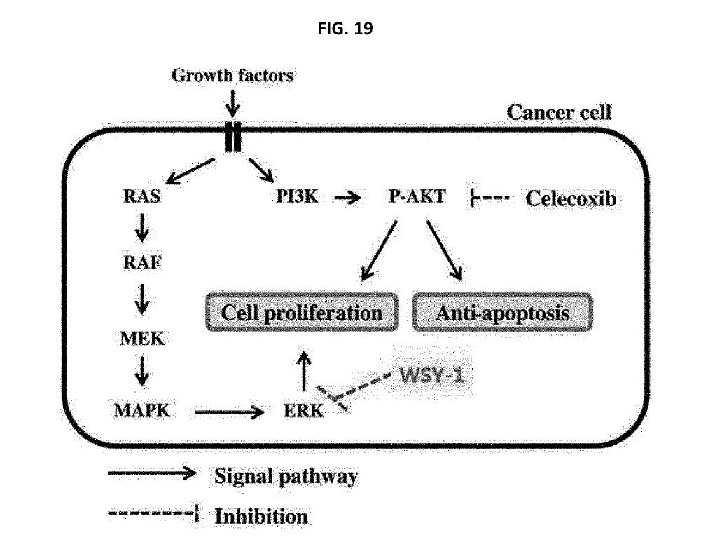

As a result of analyzing MAPK signaling pathway activated during tumorigenesis, it was found that phosphorylation of ERK is considerably reduced by WSY-1 treatment, and phosphorylation of p38 also showed a tendency to decrease. Further, it was identified that phosphorylation of JNK had not occurred, and expression of cyclin D1 as a downstream target gene of ERK was reduced. Furthermore, it was found that WSY-1 treatment did not influence on AKT activation although there was substantially no change in expression level of MMP2 (see FIG. 19).

Based on the above experimental results, it is predicted that anticancer effects of WSY-1 may be mainly attained through ERK activity inhibition, and more particularly, expression of proteins having pro-proliferation tendency may be prevented by ERK activity inhibition.

### CONCLUSION

In Vitro Efficacy Validation Aspect

According to the present study, WSY-1 and WSY-3 among the four types of natural products (WSY-0, WSY-1, WSY-3, and WSY-37) showed HCC proliferation inhibitory effects in a variety of in vitro assays.

WSY-3 induces necrosis-dependent cell death, while WSY-1 showed induction of apoptosis-dependent cell death.

In particular, it was identified that treatment using 200 μg/mL WSY-1 exhibited proliferation inhibitory effects in regard to not only different HCC cell lines but also sorafenib-resistant Huh7 cells or hyper-metastatic HCC cells, that is, HCCLM3.

In Vivo Efficacy Validation Aspect

For in vivo efficacy test, a proper HCC model was established, and appropriate dose, dosing frequency and dosing method were desirably set in order to identify in vivo anticancer effects of WSY-1. More particularly, it was identified that oral administration of WSY-1 in an amount of 100 mpk daily could significantly decrease a degree of HCC formation, as compared to an experimental group without administration of WSY-1.

Mechanism Verification Aspect

It is predicted that ant-cancer effects of WSY-1 are attained by ERK activity inhibition, and more particularly, expression of proteins having pro-proliferation tendency may be prevented by ERK activity inhibition.

Overall Discussion

Through the above experiments, it was identified that Nardostachys jatamansi extract had in vitro and in vivo hepatocellular carcinoma (HCC) inhibitory efficacies, and in particular, exhibited anticancer effects on even sorafenib-resistant HCC. Further, it was found that such anticancer effects were derived from ERK activity inhibition through MAPK signaling pathway.

Accordingly, a composition including Nardostachys jatamansi extract may have anticancer effects in regard to liver cancer, in particular, specific liver cancer resistant to an anticancer agent such as sorafenib.

1-8. (canceled)

9. A method for preventing or treating a liver cancer, the method comprising administering to a subject in need thereof a composition comprising Nardostachys jatamansi extract.

10. The method of claim 9, wherein the liver cancer is a liver cancer having resistance to an anticancer agent.

11. The method of claim 9, wherein the liver cancer is a liver cancer resistant to sorafenib.

12. The method of claim 9, wherein the liver cancer is a progressive liver cancer.

13. The method of claim 9, wherein the Nardostachys jatamansi extract is an extract in an organic solvent or a hot water.

14. The method of claim 9, wherein the Nardostachys jatamansi extract is an extract in an organic solvent selected from the group consisting of lower alcohol, acetone, chloroform, methylene chloride, ether, ethyl acetate, and hexane.

15. The method of claim 9, wherein the Nardostachys jatamansi extract is an extract in ethanol.

16. The method of claim 9, wherein the composition is included in a pharmaceutical composition.

17. The method of claim 16, wherein the pharmaceutical composition further comprises a pharmaceutically acceptable carrier.

18. The method of claim 9, wherein the composition is included in a health functional food.

19. The method of claim 9, wherein the method is for treating the liver cancer, and the subject in need thereof has the liver cancer.
